(19) United States
(12) Patent Application Publication
(10) Pub. No.: US 2004/0210100 A1
(43) Pub. Date: Oct. 21, 2004

(54) Radioactive seed with identifying emission substance used in the treatment of prostate cancer

(76) Inventors: Peter D. Grimm, Seattle, WA (US); John E. Sylvester, Edmonds, WA (US)

Correspondence Address: Jensen & Puntigam, P.S., Suite 1020, 2033 Sixth Avenue, Seattle, WA, 98121, US

(21) Appl. No.: 10/414,622

(22) Filed: Apr. 16, 2003

(51) Int. Cl. A61M 36/12

(52) U.S. Cl. 600/8

(57) ABSTRACT

The radioactive seed used in brachytherapy includes a casing of titanium and a radioactive substance element positioned within the casing, typically in the form of a band/coating, such as on a marker rod, or in the form of individual beads. The seed includes an emission element which produces emissions detectable by an MRI apparatus. The emission element can be in the form of a band/coating on a marker rod, or positioned on the casing or in the form of beads positioned within the casing.

### TECHNICAL FIELD

This invention relates generally to radioactive seed implantation for treatment of prostate cancer, and more specifically concerns a new radioactive seed which includes an emission element or substance within the seed which permits detection of the position of the seed within the prostate by a magnetic resonance imaging (MRI) apparatus.

### BACKGROUND OF THE INVENTION

Radioactive seed implantation treatment for prostate cancer, known as brachytherapy, is an increasingly popular and effective treatment. In brachytherapy, radioactive seeds are loaded into hollow needles, with the needles then being inserted into the prostate. If the radioactive seeds are separate when loaded into the needle, they remain positioned separately within the prostate when the needle is withdrawn. Alternatively, if the seeds are connected in a string arrangement, such as by an absorbable suture arrangement when loaded in the needle, they remain in such a connected relationship in the prostate when the needle is removed.

Following implanting of the seeds in the prostate, a computerized tomography (CT) scan of the prostate is typically performed. The CT scan is used to determine the actual location of the seeds within the prostate, which information is then used to perform a calculation of dose to the prostate from the seeds due to their actual position in the prostate, referred to as dosimetry. While the CT scan does in fact accurately identify the position of the seeds within the prostate, it is not particularly accurate or precise in defining the boundaries of the prostate and/or in distinguishing the prostate from surrounding bodily structures/tissues, particularly vessels and muscles. The prostate and surrounding blood vessels typically appear as a single member, rather than separate anatomical parts.

Further, the CT scan process often overestimates the size of the prostate, by as much as 20-30%. A subjective interpretation then must be made of the actual size of the prostate for dosimetry purposes. This ultimately results in an imprecise calculation of dose to the prostate from the implanted seeds, since accurate dosimetry requires an accurate knowledge of the volume of the prostate.

Alternatively, it is known that MRI images of the prostate are more accurate in defining the true size of the prostate and in separating other anatomical structures from the prostate. However, an MRI apparatus does not accurately detect the seeds themselves positioned within the prostate, and therefore MRI scans to date have not been used in postoperative dosimetry evaluations.

Attempts have been made to combine CT images with MRI images to allow for more accurate postoperative dosimetry. However, combining the two images is a complex process and is also expensive, requiring both a CT scanner and an MRI scan apparatus. Generally, it would be more desirable if an MRI apparatus could detect the position of the radioactive seeds in the prostate in addition to accurately determining the size of the prostate.

### SUMMARY OF THE INVENTION

Accordingly, the present invention is a radioactive seed for use in brachytherapy treatment of cancer, comprising: a radioactive seed which includes a casing, such as a cylinder, and an internal radioactive substance for treatment of cancer; and an emission element associated with said seed for producing emissions which are detectable by a magnetic resonance imaging MRI apparatus, such that the MRI apparatus can produce both an accurate image of the prostate and an accurate location of implanted seeds within the prostate

### BRIEF DESCRIPTION OF THE DRAWING

FIG. 1 shows a longitudinal cross-section of a conventional radioactive seed used in brachytherapy.

FIG. 2 shows one embodiment of a generic radioactive seed which incorporates the present invention.

FIG. 3 shows an alternative arrangement with the generic radioactive seed of FIG. 2.

### BEST MODE FOR CARRYING OUT THE INVENTION

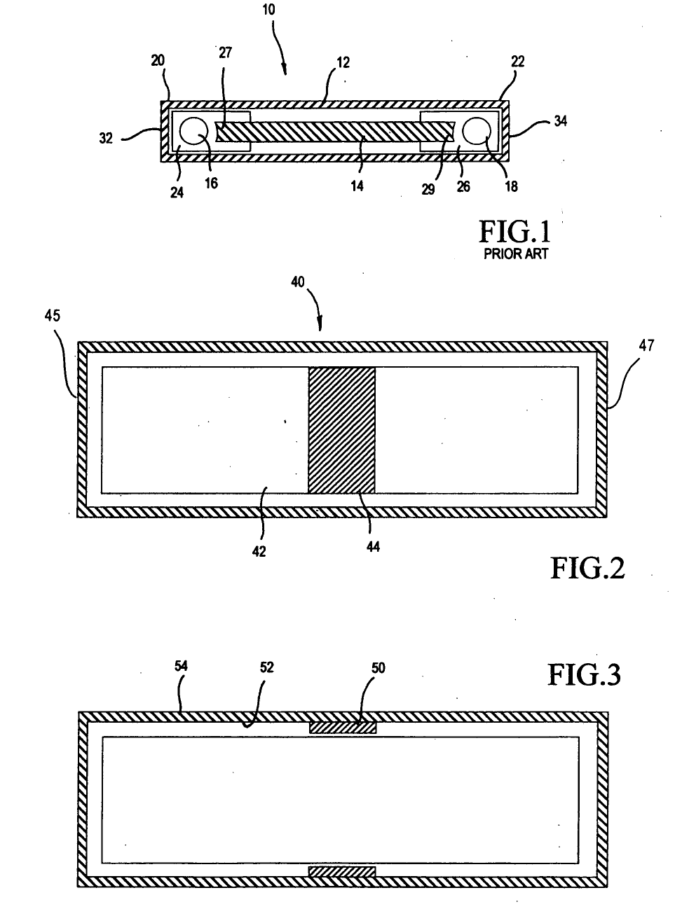

The radioactive seeds used in the brachytherapy treatment of prostate cancer can take a variety of forms. Typically, they will all include a titanium cylinder containing a radioactive substance and a marker substance. One example of such a seed is shown in FIG. 1. The seed, shown generally at 10, includes a thin-walled, open-ended titanium cylinder 12. In one embodiment, the walls of cylinder 12 are 0.05 mm thick, although this can vary. The length of the cylinder will typically be 4.4-5 mm, with a diameter of 0.8 mm, although those dimensions can also vary to some extent.

Internally of titanium cylinder 12 of FIG. 1 is a “marker” element, or substance shown in FIG. 1 as a rod 14. Rod 14 can vary in length, but in the example shown is 3 mm long with a 0.35 mm diameter. The marker rod 14 allows the seed to be “seen”, i.e. detected, by a CT scanning apparatus. Marker rod 14 can be made of various materials, including, for example, lead, silver or various alloys such as a platinum/iridium combination. Other materials can be used as well to accomplish the marker function, as long as the material is detectable by a CT scan or x-ray device.

Also positioned within titanium cylinder 12 is radioactive material, which can take various forms within the cylinder. The radioactive material is typically Palladium or Iodine 125, although it should be understood that the present invention is not limited to a particular radioactive substance. In the embodiment of FIG. 1, the radioactive substance is in the form of two beads 16 and 18. The beads in FIG. 1 are approximately 0.5 mm in diameter, although this also can be varied. The beads 16 and 18 are located approximately at opposing ends 20-22 of titanium cylinder 12. Beads 16 and 18 are captured in separate holder-type elements 24 and 26 which, for instance, can be made from a material such as silver and are configured to capture and hold the opposing ends 27-29 of marker rod 14.

The arrangement of FIG. 1, including rod 14, beads 16 and 18, and holder elements 24 and 26, is configured to be packed relatively tightly within titanium cylinder 12. After those elements are all inserted into cylinder 12, end disk portions 32 and 34 of titanium are welded to the ends of cylinder 12, forming a closed seed.

It should be understood that the seed arrangement shown in FIG. 1 is one example only, for illustration, and that seeds can take various configurations; typically they will vary from manufacturer to manufacturer to distinguish them in the marketplace. In one example of an alternative arrangement, marker rod 14 is coated at one or more locations along the length thereof with the radioactive substance, instead of the radioactive substance being in the form of individual beads such as 16 and 18. Holder elements may not be necessary in such an alternative arrangement.

As a further alternative, both the marker substance and the radioactive substance could be in the form of individual beads, with the marker beads and the radioactive substance beads being individually packed within titanium cylinder 12. The beads, could vary in size, from relatively large to quite small, such as within the range of 0.1 mm to 0.7 mm. The “beads” could take various configurations and are not limited to a spherical shape. It should be understood that the “marker substance” and “radioactive substance” portions of current commercial brachytherapy seeds could take the various arrangements and configurations described above, as well as others, in the seed of the present invention.

FIGS. 2 and 3 show two possible arrangements of the radioactive seed of the present invention. In FIG. 2, the radioactive 40 seed includes a titanium cylinder 41 of conventional size and dimensions, as discussed above. Positioned in cylinder 40 are a radioactive substance and/or marker substance element in the form of a rod 42. A band 44 of proton-emitting material is coated on the exterior of rod 42. One example of such material is Gadolidium. An MRI apparatus is capable of detecting the proton-emissions of Gadolidium, enabling the MRI apparatus to identify the location of the seeds in the prostate, such as for dosimetry. While the band 44 shown in FIG. 2 is shown as a coating about a portion of the rod 42 near the center thereof, the band could be at various locations, along the rod or could extend for substantially the entire length of the rod.

In the embodiment shown, band 44 is 1.0 mm inches wide and 0.6 mm inches thick, although this can be varied. While in the embodiment shown, a proton-emitter such as Gadolidium is used, other proton or other emission substances are possible; the present invention is not limited to Gadolidium. The material must, however, produce emissions which are readily detectable by an MRI device or produce enhanced MRI imaging. Substances which may be useful in enhancing the MRI image include supraparamagnetic ion oxide and a combination of manganese, organic ligand and fodipir, sometimes known as mangafodipir.

As an alternative to the arrangement shown in FIG. 2, the proton-emission substance could take the form of small beads as part of rod 42 or the form of bead(s) in combination with radioactive substance beads and marker beads, all packed into a seed cylinder, such as cylinder 40. In each of the above cases, end portions 45, 47 for the cylinder are then welded into place to finalize the seed product.

In addition, referring to FIG. 3, the proton-emission substance could be in the form of a band or coating 50 located on an internal surface 52 of a titanium cylinder seed 54, or in still another alternative could be in the form of a coating on an external surface of the seed cylinder. Further, proton emission beads could be embedded in the titanium cylinder itself. Accordingly, it should be understood that the proton-emission elements could take a wide variety of forms and arrangements in combination with the various seed arrangements.

Accordingly, a new brachytherapy seed arrangement has been disclosed which includes an emission-producing element, the emissions of which are detectable by a magnetic resonance imaging (MRI) machine. With such an arrangement, the MRI apparatus can be conveniently and appropriately used for postoperative dosimetry functions, as it not only will have the capability of producing an accurate and true image of the prostate relative to the surrounding tissues and vessels, an important advantage over CT scanning devices, but also can readily detect the position of the individual seeds which have been implanted in the prostate.

This permits a more accurate postoperative study and evaluation of the quality of the implant seeds. It will also permit adjustments/improvements to the basic brachytherapy technique, particularly in precision and efficiency improvements and ultimately better cancer control rates. Furthermore, the use of such an emission element can be incorporated in a wide variety of existing and future brachytherapy seed arrangements. This is important, since each commercial manufacturer of radioactive seeds has its own particular seed configuration and arrangement to create its own brand identity.

Although a preferred embodiment of the invention has been described for purposes of illustration, it should be understood that various changes, modifications and substitutions might be incorporated in the embodiment without departing from the spirit of the invention, which is defined by the claims which follow.

What is claimed is:

1. A radioactive seed for use in brachytherapy treatment of cancer, comprising:
a radioactive seed which includes a casing and an internally positioned radioactive substance element for treatment of cancer; and
an emission element associated with said seed for producing emissions which are detectable by a magnetic resonance imaging (MRI) apparatus, such that the MRI apparatus can provide both an accurate image of the prostate and an accurate location of implanted seeds within the prostate.

2. An article of claim 1, wherein the seed casing is titanium and the radioactive substance is iodine 125.

3. An article of claim 1, wherein the emission element is a proton-emitting element.

4. An article of claim 2, wherein the emission element is a proton-emitting element.

5. An article of claim 4, wherein the proton-emitting element is Gadolidium.

6. An article of claim 1, wherein the radioactive substance is in the form of individual beads.

7. An article of claim 1, wherein the emission element is in the form of a coating on a radioactive substance/marker rod member within the casing.

8. An article of claim 1, wherein the emission element is in the form of at least one bead positioned within the casing.

9. An article of claim 1, wherein the emission element is in the form of a coating on the seed casing.
